(19) United States
(12) Patent Application Publication
(10) Pub. No.: US 2002/0045869 A1
(43) Pub. Date: Apr. 18, 2002

(54) Absorbent composites comprising superabsorbent materials

(76) Inventors: Richard Norris Dodge II, Appleton, WI (US); Sridhar Ranganathan, Suwanee, GA (US); Sandra Marie Yarbrough, Menasha, WI (US); Wendy Lynn Van Dyke, Appleton, WI (US); Michael John Niemeyer, Appleton, WI (US); Yong Li, Appleton, WI (US)

Correspondence Address: KILPATRICK STOCKTON LLP, Attn: John S. Pratt, Suite 2800, 1100 Peachtree Street, Atlanta, GA, 30309, US

(21) Appl. No.: 09/900,512

(22) Filed: Jul. 6, 2001

Related U.S. Application Data
(63) Continuation-in-part of application No. 09/475,830, filed on Dec. 30, 1999.
(60) Provisional application No. 60/114,332, filed on Dec. 31, 1998.

(51) Int. Cl. C09K 3/00; A61F 13/20; A61F 13/15; D04H 3/00; D04H 1/00; C01B 31/16

(52) U.S. Cl. 604/368; 252/184; 604/378; 442/340

(57) ABSTRACT

The present invention is directed to absorbent composites having enhanced intake properties. The absorbent composites have a Composite Permeability (CP) value at full swelling of greater than about 100×10⁻⁸ cm². Further, the absorbent composites have a Composite Permeability/3^{rd} Insult Fluid Intake Flowback Evaluation (FIFE) intake relationship, which results in enhanced intake properties. The present invention also provides a method of making absorbent composites having enhanced intake properties. The present invention further provides absorbent composites and their applicability in disposable personal care products. The present invention utilizes superabsorbent materials having a Gel Bed Permeability (GBP) value of greater than about 70×10⁻⁹ cm² and an Absorbency Under Load (AUL) value at 0.6 psi of less than about 25 g/g.

### CROSS-REFERENCE TO RELATED APPLICATIONS

This application is a continuation-in-part application of U.S. Ser. No. 09/475,830, filed Dec. 30, 1999, which claims the benefit of U.S. Provisional Application Ser. No. 60/114,332, filed on Dec. 31, 1998.

### FIELD OF THE INVENTION

The present invention is directed to absorbent articles containing superabsorbent materials. The present invention is also directed to a method of making absorbent articles containing superabsorbent materials. The present invention is further directed to fiber-containing fabrics and webs comprising superabsorbent materials and their applicability in disposable personal care products.

### BACKGROUND OF THE INVENTION

In the manufacture of disposable diapers, there is continual effort to improve the performance characteristics of the diaper. Although the structure of a diaper has many components, in many instances the in-use performance of the diaper is directly related to the characteristics of the absorbent composite contained within the diaper. Accordingly, diaper manufacturers strive to find ways of improving the properties of the absorbent composite, including in-use absorbency, in order to reduce the tendency of the diaper to leak.

One means of reducing the leakage of a diaper has been the extensive use of superabsorbent materials. Recent trends in commercial diaper designs have been to use more superabsorbent materials and less fiber in order to make the diaper thinner. However, notwithstanding the increase in total absorbent capacity contributed by the addition of larger amounts of superabsorbent material, such diapers often still suffer from excessive leaking during use.

One reason that diapers with a high content of superabsorbent materials still leak is that many superabsorbent materials are unable to absorb liquid at the rate at which the liquid is applied to the absorbent composite during use. The addition of fibrous material to the absorbent composite improves the leakage of an absorbent composite by temporarily holding the liquid until the superabsorbent material absorbs it. Fibers also serve to separate the particles of superabsorbent material so that gel-blocking does not occur. As used herein, the term “gel-blocking” refers to the situation wherein particles of superabsorbent material deform during swelling and block the interstitial spaces between the particles, or between the particles and the fibers, thus preventing the flow of liquid through the interstitial spaces. Even when fibrous material is incorporated into an absorbent composite, a poor choice of a superabsorbent material, especially one which exhibits gel-blocking behavior within the absorbent composite, results in poor liquid handling properties initially and later in the life cycle of the absorbent composite. Consequently, the choice of a particular superabsorbent material greatly affects the in-use absorbency and leakage of the absorbent product.

Another problem with commercially available diapers is the tendency of diapers to leak after multiple insults. As used herein, the term “insults” refers to a single introduction of liquid into the absorbent composite or diaper. During use, a diaper is typically exposed to multiple insults during the life cycle of the diaper. To reduce diaper leakage during the life cycle of the diaper, it is desirable to maintain the level of intake performance of the absorbent composite throughout the life of the product.

A number of U.S. patents address different problems associated with absorbent composites. For example, U.S. Pat. No. 5,147,343 issued to Kellenberger teaches the importance of having a superabsorbent with high Absorbency Under Load values in an absorbent product. U.S. Pat. No. 5,149,335 issued to Kellenberger et al. teaches the importance of superabsorbent rate and capacity in a composite. U.S. Pat. No. 5,415,643 issued to Kolb teaches a method of increasing the flushability of an absorbent composite by incorporating superabsorbent materials having a high Absorbency Under Load (AUL) to Centrifuge Retention Capacity (CRC) ratio with AUL evaluated in 90 minutes under 0.6 psi (41,370 dynes/cm²) into the composite. U.S. Pat. No. 5,415,643 issued to Melius et al. teaches the importance of AUL values under different pressures. U.S. Pat. No. 5,599,335 issued to Goldman emphasizes the benefits of the combination of high Saline Flow Conductivity and high Performance Under Pressure. U.S. Pat. No. 5,728,082 issued to Gustafsson et al describes an absorbent body consisting of two layers containing superabsorbent, wherein the superabsorbent in the first layer has a high degree of cross-linking while the superabsorbent in the second layer has a higher absorbent capacity than the superabsorbent in the first layer.

The aforementioned patents disclose specific superabsorbent properties, which result in improved composite performance. In general, the aforementioned patents teach that superabsorbent materials exhibiting high capacity under load result in improved gel stiffness and permeability behavior for enhanced composite performance. However, the aforementioned patents do not specifically address the problems mentioned above, namely, improving leakage/intake over the life cycle of the absorbent composite.

What is needed in the art is a method of determining which superabsorbent materials lead to optimum composite properties. What is also needed in the art is an absorbent composite containing superabsorbent materials, which exhibits improved fluid intake rate, and superior fluid intake of multiple insults over the life of the composite, without the problems associated with known absorbent composites.

### SUMMARY OF THE INVENTION

The present invention is directed to absorbent composites containing superabsorbent materials, which have been developed to address the above-described problems associated with currently available, absorbent composites and other absorbent composites described in literature. The absorbent composites contain superabsorbent materials, which have a Gel Bed Permeability (GBP) value of greater than about 70×10⁻⁹ cm² and an Absorbency Under Load (AUL) value of less than about 25 g/g at 0.6 psi (41,370 dynes/cm²). This combination of properties for superabsorbent materials enables an absorbent composite to have improved fluid intake rate and superior fluid intake of multiple insults over the life of the composite. Unlike known absorbent composites, which lose their fluid intake performance over the life of the composite, the absorbent composites of the present invention perform exceptionally well, exhibiting superior fluid intake after multiple insults to the composite.

The present invention is also directed to a method of making absorbent articles containing superabsorbent materials having a Gel Bed Permeability (GBP) value of greater than about 70×10⁻⁹ cm² and an Absorbency Under Load (AUL) value of less than about 25 g/g at 0.6 psi (41,370 dynes/cm²). The superabsorbent materials may be incorporated into a fibrous substrate by a variety of processes. The superabsorbent material may be incorporated into a fibrous substrate as solid particulate material or as a solution. The superabsorbent materials may be in any form suitable for use in absorbent composites including particles, fibers, flakes, spheres, and the like.

The present invention is further directed to absorbent composites comprising superabsorbent materials and fibrous material, and their applicability in disposable personal care products. The absorbent composites of the present invention are particularly useful as absorbent components in personal care products such as diapers, feminine pads, panty liners, incontinence products, and training pants.

### BRIEF DESCRIPTION OF THE FIGURES

FIG. 1 is an illustration of equipment for determining the Gel Bed Permeability (GBP) value of a superabsorbent material.

FIG. 2 is an cross-sectional view of the piston head taken along line 2-2 of FIG. 1.

FIG. 3 is an illustration of equipment for determining the Absorbency Under Load (AUL) value of a superabsorbent material.

FIG. 4 is an cross-sectional view of the porous plate taken along line 4-4 of FIG. 3.

FIGS. 5a-c are an illustration of equipment for determining the Composite Permeability value of an absorbent composite.

FIG. 6 is an illustration of equipment for determining the Fluid Intake Flowback Evaluation (FIFE) value of an absorbent composite.

FIG. 7 is an illustration of equipment for determining the Intake/Desorption value of an absorbent composite.

FIG. 8 is a graphical representation of the data set forth in Table 10 showing that the composite permabilities of the present invention are much higher at the same intake rates than the prior art.

### DESCRIPTION OF THE PREFERRED EMBODIMENTS

The present invention is directed to absorbent composites containing superabsorbent materials, wherein the absorbent composites possess the ability to maintain exceptional intake performance even after multiple insults to the composite. The present invention achieves these results by approaching the problems of intake performance and leakage in an unconventional manner. Traditionally, the approach taken to address fluid intake has been to incorporate superabsorbents having a high capacity under load into an absorbent composite. The goal was to produce an absorbent composite having increased capacity and permeability behavior, and ultimately provide to the composite improved intake performance over multiple insults. However, it has been determined that the pursuit of higher superabsorbent capacity inevitably leads to limited performance improvement. Instead, the present invention achieves high composite permeability and other desirable composite properties using lower capacity superabsorbents.

As used herein, the term “superabsorbent material” refers to a water-swellable, water-insoluble organic or inorganic material capable, under the most favorable conditions, of absorbing more than 15 times its weight in an aqueous solution containing 0.9 weight percent sodium chloride. Organic materials suitable for use as a superabsorbent material of the present invention may include natural materials such as agar, pectin, guar gum, and the like; as well as synthetic materials, such as synthetic hydrogel polymers. Such hydrogel polymers include, but are not limited to, alkali metal salts of polyacrylic acids, polyacrylamides, polyvinyl alcohol, ethylene maleic anhydride copolymers, polyvinyl ethers, hydroxypropylcellulose, polyvinylmorpholinone; and polymers and copolymers of vinyl sulfonic acid, polyacrylates, polyacrylamides, polyvinylpyrridine, and the like. Other suitable polymers include hydrolyzed acrylonitrile grafted starch, acrylic acid grafted starch, and isobutylene maleic anhydride copolymers and mixtures thereof. The hydrogel polymers are desirably lightly crosslinked to render the material substantially water insoluble. Crosslinking may, for example, be by irradiation or by covalent, ionic, van der Waals, or hydrogen bonding. The superabsorbent materials may be in any form suitable for use in absorbent composites including particles, fibers, flakes, spheres, and the like.

While a wide variety of superabsorbent materials are known, the present invention relates, in one aspect, to the proper selection of superabsorbent materials to allow formation of improved absorbent composites and disposable absorbent garments. The present invention is directed to a method of achieving optimum performance in an absorbent composite due to the discovery that superabsorbent materials having a high Gel Bed Permeability (GBP) value and a low Absorbency Under Load (AUL) value at 0.6 psi (41,370 dynes/cm²) provide unexpected intake performance improvement over known superabsorbent materials. More specifically, superabsorbent materials having, in combination, a Gel Bed Permeability (GBP) value of greater than about 70×10⁻⁹ cm² and an Absorbency Under Load (AUL) value of less than about 25 g/g at 0.6 psi (41,370 dynes/cm²), provide desirable properties and performance to absorbent composites. These lower capacity superabsorbent materials have the capability of delivering improved intake performance as described below.

The present invention has determined that lower capacity superabsorbent materials provide much more room for improvement in absorbent composite performance. Not only do the low capacity superabsorbent materials enhance the ability of the absorbent composite to rapidly take in liquid, the low capacity superabsorbent materials also enable constant or even improved fluid intake performance over the life of the absorbent composite.

The present invention discloses that superabsorbent materials may be divided into two categories: those having a (1) Gel Bed Permeability (GBP) value of greater than about 70×10⁻⁹ cm² and an Absorbency Under Load (AUL) value of less than about 25 g/g at 0.6 psi (41,370 dynes/cm²) (Class-I superabsorbents), and the rest (Class-II superabsorbents). Use of Class I superabsorbents having relatively low AUL behavior and high GBP provides the unexpectedly improved intake behavior described below.

Superabsorbent materials suitable for the present invention may include any superabsorbent material, which has a Gel Bed Permeability (GBP) value of greater than about 70×10⁻⁹ cm² and an Absorbency Under Load (AUL) value of less than about 25 g/g at 0.6 psi (41,370 dynes/Cm²). Desirably, the superabsorbent material has a GBP value of greater than about 150×10⁻⁹ cm² and an AUL value of less than about 25 g/g at 0.6 psi. More desirably, the superabsorbent material has a GBP value of greater than about 180×10⁻⁹ cm² and an AUL value of less than about 25 g/g at 0.6 psi. More desirably, the superabsorbent material has a GBP value of greater than about 210×10⁻⁹ cm² and an AUL value of less than about 25 g/g at 0.6 psi. More desirably, the superabsorbent material has a GBP value of greater than about 250×10⁻⁹ cm² and an AUL value of less than about 25 g/g at 0.6 psi. Even more desirably, the superabsorbent material has a GBP value of greater than about 300×10⁻⁹ cm² and an AUL value of less than about 25 g/g at 0.6 psi.

In another desired embodiment of the present invention, the superabsorbent material has a GBP value of greater than about 70×10⁻⁹ cm² and an AUL value of less than about 24 g/g at 0.6 psi. More desirably, the superabsorbent material has a GBP value of greater than about 150×10⁻⁹ Cm² and an AUL value of less than about 24 g/g at 0.6 psi. More desirably, the superabsorbent material has a GBP value of greater than about 180 ×10⁻⁹ cm² and an AUL value of less than about 24 g/g at 0.6 psi. More desirably, the superabsorbent material has a GBP value of greater than about 210×10⁻⁹ cm² and an AUL value of less than about 24 g/g at 0.6 psi. More desirably, the superabsorbent material has a GBP value of greater than about 250×10⁻⁹ cm² and an AUL value of less than about 24 g/g at 0.6 psi. Even more desirably, the superabsorbent material has a GBP value of greater than about 300×10⁻⁹ cm² and an AUL value of less than about 24 g/g at 0.6 psi.

In a further desired embodiment of the present invention, the superabsorbent material has a GBP value of greater than about 70×10⁻⁹ em² and an AUL value of less than about 23 g/g at 0.6 psi. More desirably, the superabsorbent material has a GBP value of greater than about 150×10⁻⁹ cm² and an AUL value of less than about 23 g/g at 0.6 psi. More desirably, the superabsorbent material has a GBP value of greater than about 180 ×10⁻⁹ cm² and an AUL value of less than about 23 g/g at 0.6 psi. More desirably, the superabsorbent material has a GBP value of greater than about 210×10⁻⁹ cm² and an AUL value of less than about 23 g/g at 0.6 psi. More desirably, the superabsorbent material has a GBP value of greater than about 250×10⁻⁹ cm² and an AUL value of less than about 23 g/g at 0.6 psi. Even more desirably, the superabsorbent material has a GBP value of greater than about 300×10⁻⁹ cm² and an AUL value of less than about 23 g/g at 0.6 psi.

In yet a further desired embodiment of the present invention, the superabsorbent material has a GBP value of greater than about 70×10⁻⁹ cm² and an AUL value of less than about 21 g/g at 0.6 psi. More desirably, the superabsorbent material has a GBP value of greater than about 150×10⁻⁹ cm² and an AUL value of less than about 21 g/g at 0.6 psi. More desirably, the superabsorbent material has a GBP value of greater than about 180 ×10⁻⁹ cm² and an AUL value of less than about 21 g/g at 0.6 psi. More desirably, the superabsorbent material has a GBP value of greater than about 210×10⁻⁹ cm² and an AUL value of less than about 21 g/g at 0.6 psi. More desirably, the superabsorbent material has a GBP value of greater than about 250×10⁻⁹ cm² and an AUL value of less than about 21 g/g at 0.6 psi. Most desirably, the superabsorbent material has a GBP value of greater than about 300×10⁻⁹ cm² and an AUL value of less than about 21 g/g at 0.6 psi.

In addition to having a GBP value of greater than about 70×10⁻⁹ cm² and an AUL value of less than about 25 g/g at 0.6 psi, desirably the superabsorbent material used in the present invention has a pH in a range such that no skin irritation can occur when the superabsorbent material is present in an absorbent composite. Desirably, the superabsorbent material used in the present invention has a pH of from about 3 to about 8, as measured by the pH test method described below. More desirably, the superabsorbent material used in the present invention has a pH of from about 4 to about 7. Most desirably, the superabsorbent material used in the present invention has a pH of from about 5.2 to about 6.5.

In one embodiment of the present invention, the superabsorbent material comprises a sodium salt of a cross-linked polyacrylic acid. Suitable superabsorbent materials include, but are not limited to, Stockhausen W-65431 (available from Stockhausen Chemical Company, Inc., Greensboro, N.C.); Dow AFA-173-60A, Dow AFA-173-60B, Dow XU 40671.00, Dow XUS 40665.07, Dow XZ-91060.02/91080.20 (hereinafter, “Dow XZ”), and Dow XUS 40667.01 (all available from The Dow Chemical Company, Midland, Mich.).

The present invention is further directed to absorbent composites containing one or more Class I superabsorbent materials described above. The Class I superabsorbent materials may be used alone or in combination with one or more Class II superabsorbent materials. These absorbent composites offer increased permeability as compared to known absorbent composites.

The absorbent composites of the present invention desirably possess constant or improved fluid intake over the life of the composite. The fundamental absorbent property of composite permeability of an absorbent material is a key to fast intake. One method of measuring composite permeability is with the Composite Permeability test, which is described in detail below. This test measures the time required for a fixed volume of liquid to flow through a pre-saturated composite in the z-direction. As shown in Table 1, the majority of Class-I superabsorbent materials enable about twice as much composite permeability for an absorbent composite containing 50 wt % superabsorbent material and 50 wt % wood pulp fibers as compared to control superabsorbent materials, Favor 880 (available from Stockhausen Inc., Greensboro, N.C.) and Dow DryTech 2035 (available from Dow Chemical Company, Midland, Mich.).

TABLE 1
Composite Permeability for Absorbent Composites
Containing 50 wt % Fibers and 50 wt % SAM

| | | Composite Permeability |
|---|---|---|
| Class | Superabsorbent Material | (×10⁻⁸ cm²) |
| I | W-65431 | ˜191 |
| I | AFA-173-60A | ˜100 |
| I | AFA-173-60B | ˜177 |
| I | XUS 40665.07 | ˜202 |
| I | XU 40671.00 | ˜192 |
| I | XZ | ˜115 |
| I | XUS 40667.01 | ˜168 |
| II | Favor 880 | ˜112 |
| II | DryTech 2035 | ˜61 |

Another important measure of intake performance is measured by the Fluid Intake Flowback Evaluation (FIFE) test, which is described in detail below. The FIFE test measures how fast liquid can flow into a material. Table 2 shows the 3^{rd} insult FIFE intake rates for a variety of absorbent composites containing 50 wt % superabsorbent material and 50 wt % wood pulp fibers. It can be seen that absorbent composites containing different superabsorbents exhibit different FIFE intake rates. However, FIFE intake rates and composite permeabilities may also be manipulated by changing the type of fiber used. As shown in Table 3, most of the Class-I superabsorbents exhibited fast intake rates (>2.75 ml/sec).

TABLE 2
3^{rd} Insult FIFE Rate for 50 wt % SAM Absorbent Composites

| | | 3^{rd} Insult FIFE |
|---|---|---|
| | Superabsorbent | Rate |
| Class | Material | (ml/sec) |
| I | W-65431 | ˜3.2 |
| I | AFA-173-60A | ˜2.5 |
| I | AFA-173-60B | ˜3.1 |
| I | XUS 40665.07 | ˜3.1 |
| I | XU 40671.00 | ˜3.4 |
| I | XZ | ˜2.1 |
| I | XUS 40667.01 | ˜3.0 |
| II | Favor 880 | ˜2.1 |
| II | DryTech 2035 | ˜1.6 |

The improved intake behavior as seen by the 3^{rd} Insult FIFE Intake Rate may be controlled by the type and amount of superabsorbent material present in the absorbent composite. Table 3 shows the 3^{rd} Insult FIFE Intake Rate for two sets of composites containing either a conventional superabsorbent material (identified as Favor 880) or a Class I superabsorbent material, exhibiting the desirable properties of a GBP value greater than about 70×10⁻⁹ cm² and an AUL value of less than about 25 g/g at 0.6 psi (identified as XUS 40665.07). Additionally, for each type of superabsorbent material, composites containing either 30, 40, 50, or 60 wt % superabsorbent material, with the remainder wood pulp fiber, were prepared and evaluated. All composites had a total basis weight of 400 gsm. This results in composites having a superabsorbent basis weight of 120, 160, 200, or 240 gsm.

TABLE 3
3^{rd} Insult FIFE Rate for SAM Absorbent Composites with
Variable SAM Weight Percent

3^{rd} Insult FIFE Rate
(ml/sec)

| Superabsorbent | Superabsorbent | Favor | XUS |
|---|---|---|---|
| wt % | Basis Weight (gsm) | 880 | 40665.07 |
| 30 | 120 | 6.6 | 6.6 |
| 40 | 160 | 4 | 5.7 |
| 50 | 200 | 2.2 | 3.1 |
| 60 | 240 | 2 | 3 |

As can be seen in Table 3, as the amount of superabsorbent material in the composite changes, the 3^{rd} Insult FIFE Intake Rate of the composite changes. In addition, at 40, 50, and 60 wt % superabsorbent levels, a composite comprising Class I superabsorbent material (XUS 40665.07) exhibits a faster, more desirable, 3^{rd} Insult FIFE Intake Rate compared to a composite comprising a superabsorbent that is not Class I (Favor 880).

To further demonstrate the impact of the type and amount of superabsorbent material present in the composite on the composite intake behavior, Table 4 shows the 3^{rd} Insult FIFE Intake Rate for two sets of composites containing either a conventional superabsorbent (identified as Favor 880) or a Class I superabsorbent material, exhibiting the desirable properties of a GBP value greater than about 70×10⁻⁹ cm² and an AUL value of less than about 25 g/g at 0.6 psi (identified as XUS 40665.07). However, in these two sets, for each type of superabsorbent material, composites having a total composite basis weight of either 200, 300, 400, or 500 gsm were prepared and evaluated. All composites had 50 wt % superabsorbent material and 50% wood pulp fiber. This results in composites having a superabsorbent basis weight of 100, 150, 200, or 250 gsm.

TABLE 4
3^{rd} Insult FIFE Rate for 50 wt % SAM Absorbent
Composites with Variable Composite Basis Weight

3^{rd} FIFE Rate
(ml/sec)

| Composite Basis | Superabsorbent | Favor | XUS |
|---|---|---|---|
| Weight (gsm) | Basis Weight (gsm) | 880 | 40665.07 |
| 200 | 100 | 4.4 | 4.4 |
| 300 | 150 | 3.3 | 5.5 |
| 400 | 200 | 2.2 | 3.3 |
| 500 | 250 | 2.5 | 3.5 |

As can be seen in Table 4, as the composite basis weight (and superabsorbent basis weight) changes, the 3^{rd} Insult FIFE Intake Rate of the composite changes. In addition, at superabsorbent basis weights of 150, 200, or 250 gsm, a composite comprising Class I superabsorbent material (XUS 40665.07) exhibits a faster, more desirable, 3^{rd} Insult FIFE Intake Rate compared to a composite comprising a superabsorbent that is not Class I (Favor 880). However, FIFE intake rates and composite permeabilities may also be manipulated by changing the type of fiber used.

As would be expected, the composite permeability may also be controlled by the amount of superabsorbent material present in the absorbent composite. Table 5 shows the composite permeability for two sets of composites containing one of two superabsorbent materials (identified as Stockhausen Favor 880 and Dow XUS 40665.07). For each type of superabsorbent material, composites containing either 30, 40, 50, or 60 wt % superabsorbent material were prepared and evaluated. All composites had a total basis weight of 400 gsm. This results in composites having a superabsorbent basis weight of 120, 160, 200, or 240 gsm.

TABLE 5
Composite Permeability for Absorbent Composites
with Variable SAM Weight Percent

| | | | | Composite | |
|---|---|---|---|---|---|
| | | | | Permeability | |
| | | | | (×10⁻⁸ cm²) | |
| | | | SAM Basis | | Dow XUS |
| Composite | Fibrous | SAM | Weight | Favor | 40665.07 |
| Sample | Material | wt % | (gsm) | 880 | (D3) |
| 1 | Wood Pulp | 30 | 120 | 161 | 226 |
| 2 | Wood Pulp | 40 | 160 | 172 | 255 |
| 3 | Wood Pulp | 50 | 200 | 112 | 202 |
| 4 | Wood Pulp | 60 | 240 | 63 | 159 |

As can be seen in Table 5, as the amount of superabsorbent material in the composite changes, the composite permeability of the composite changes. In addition, at 30, 40, 50, and 60 wt % superabsorbent levels, a composite comprising Dow XUS 40665.07 superabsorbent material exhibits a higher composite permeability compared to a composite comprising Stockhausen Favor 880 superabsorbent material.

The absorbent composites of the present invention achieve a rapid intake rate by increasing the permeability of the composite. Increases in the composite permeability lead to an increase in the intake rate. The present invention provides higher intake rates and composite permeabilities than prior art composites. This behavior is achieved through the use of the desired superabsorbents and fibers. As a result, these composites are unconventional in that the exhibit higher permeability, but with a lower capacity. Higher capacity materials, however, are unable to utilize these capacities as the composite permeabilities are much lower. Accordingly, the present invention desire higher intake rates and composite permeabilities and the composite permeabilities may be defined in terms of the intake rates as set forth below.

The absorbent composites of the present invention have a Composite Permeability value at full swelling of greater than about 100×10⁻⁸ cm². Desirably, the absorbent composites of the present invention have a CP value at full swelling of greater than about 175×10⁻⁸ cm². More desirably, the absorbent composites of the present invention have a CP value at full swelling of greater than about 190×10⁻⁸ cm². Even more desirably, the absorbent composites of the present invention have a CP value at full swelling of greater than about 205×10⁻⁸ cm². Most desirably, the absorbent composites of the present invention have a CP value at full swelling of greater than about 225×10⁻⁸ cm². When the absorbent composites of the present invention have a CP value at full swelling of greater than about 175×10⁻⁸ cm², desirably the 3^{rd} Insult FIFE intake rate is greater than about 2.00 ml/sec. More desirably, when the absorbent composites of the present invention have a CP value at full swelling of greater than about 175×10⁻⁸ cm², the 3^{rd} Insult FIFE intake rate is greater than about 2.50 ml/sec. Even more desirably, when the absorbent composites of the present invention have a CP value at full swelling of greater than about 175×10⁻⁸ cm², the 3^{rd} Insult FIFE intake rate is greater than about 2.75 ml/sec. Most desirably, when the absorbent composites of the present invention have a CP value at full swelling of greater than about 175×10⁻⁸ cm², the 3^{rd} Insult FIFE intake rate is greater than about 3.00 ml/sec.

In one embodiment, the absorbent composites of the present invention have a Composite Permeability value/3^{rd} Insult Fluid Intake Flowback Evaluation (FIFE) intake rate relationship, wherein the Composite Permeability value varies depending on the 3^{rd} Insult FIFE intake rate. Desirably, when the IR value of the absorbent composite is greater than 0 ml/sec and less than about 3.00 ml/sec, the CP value is given by the following equation:

CP≧{135−[(35/3)×(3.00×IR)]}×10⁻⁸;

when the IR value of the absorbent composite is greater than about 3.00 ml/sec and less than about 3.70 ml/sec, the CP value is given by the following equation:

CP≧{175−[(400/7)×(3.70−IR)]}×10⁻⁸; and

when the IR value of the absorbent composite is greater than about 3.70 ml/sec, the CP value is greater than or equal to 175×10⁻⁸, wherein CP has units of cm².

For example, when the absorbent composite has a 3^{rd} Insult FIFE intake rate (IR) of 3.00 ml/sec, the CP value is desirably equal to or greater than about 135×10⁻⁸ cm². Further, when the absorbent composite has a 3^{rd} Insult FIFE intake rate (IR) of 1.00 ml/sec, the CP value is desirably equal to or greater than habout 112×10⁻⁸ cm².

In yet a further embodiment, the absorbent composites of the present invention have a 3^{rd} Insult FIFE intake rate (IR) greater than 3.00 mi/sec and less than about 3.70 ml/sec. When the IR value of the absorbent composite is greater than 0 ml/sec and less than about 3.00 ml/sec, the CP value is given by the following equation:

CP≧{150−[(35/3)×(3.00−IR)]}×10⁻⁸;

when the IR value of the absorbent composite is greater than about 3.00 ml/sec and less than about 3.70 ml/sec, the CP value is given by the following equation:

CP≧{190−[(400/7)×(3.70−IR)]}×10⁻⁸; and

when the IR value of the absorbent composite is greater than about 3.70 ml/sec, the CP value is greater than or equal to 190×10⁻⁸, wherein CP has units of cm².

For example, when the absorbent composite has a 3^{rd} Insult FIFE intake rate (IR) of 3.50 ml/sec, the CP value is desirably equal to or greater than about 164×10⁻⁸ cm². Further, when the absorbent composite has a 3^{rd} Insult FIFE intake rate (IR) of 3.20 ml/sec, the CP value is desirably equal to or greater than about 146×10⁻⁸ cm².

In another embodiment, the absorbent composites of the present invention have a Composite Permeability value/3^{rd} Insult Fluid Intake Flowback Evaluation (FIFE) intake rate relationship represented by the following equations. When the IR value of the absorbent composite is greater than 0 ml/sec and less than about 3.00 ml/sec, the CP value is given by the following equation:

CP≧{165−[(35/3)×(3.00−IR)]}×10⁻⁸;

when the IR value of the absorbent composite is greater than about 3.00 ml/sec and less than about 3.70 ml/sec, the CP value is given by the following equation:

CP≧{205−[(400/7)×(3.70−IR)]}×10⁻⁸; and

when the IR value of the absorbent composite is greater than about 3.70 ml/sec, the CP value is greater than or equal to 205×10⁻⁸, wherein CP has units of cm².

In addition to the superabsorbent materials described above, the absorbent composites of the present invention may comprise means to contain the superabsorbent material. Any means capable of containing the above-described superabsorbent materials, which means is further capable of being located in a disposable absorbent garment, is suitable for use in the present invention. Many such containment means are known to those skilled in the art. For example, the containment means may comprise a fibrous matrix such as an air-laid or wet-laid web of cellulosic fibers, a meltblown web of synthetic polymeric fibers, a spunbonded web of synthetic polymeric fibers, a coformed matrix comprising cellulosic fibers and fibers formed from a synthetic polymeric material, air-laid heat-fused webs of synthetic polymeric material, open-celled foams, and the like.

Alternatively, the containment means may comprise two layers of material which are joined together to form a pocket or compartment, more particularly a plurality of pockets, which pocket contains the superabsorbent material. In such a case, at least one of the layers of material should be water-pervious. The second layer of material may be water-pervious or water-impervious. The layers of material may be cloth-like wovens and nonwoven, closed or open-celled foams, perforated films, elastomeric materials, or may be fibrous webs of material. When the containment means comprises layers of material, the material should have a pore structure small enough or tortuous enough to contain the majority of the superabsorbent material. The containment means may also comprise a laminate of two layers of material between which the superabsorbent material is located and contained. Further, the containment means may comprise a support structure, such as a polymeric film, on which the superabsorbent material is affixed. The superabsorbent material may be affixed to one or both sides of the support structure, which may be water-pervious or water-impervious.

Desirably, the absorbent composites of the present invention comprise superabsorbent material in combination with a fibrous matrix containing one or more types of fibrous materials. The fibrous material forming the absorbent composites of the present invention may be selected from a variety of materials including natural fibers, synthetic fibers, and combinations thereof. A number of suitable fiber types are disclosed in U.S. Pat. No. 5,601,542, assigned to Kimberly-Clark Corporation, the entirety of which is incorporated herein by reference. The choice of fibers depends upon, for example, the intended end use of the finished absorbent composite. For instance, suitable fibrous materials may include, but are not limited to, natural fibers such as cotton, linen, jute, hemp, wool, wood pulp, etc. Similarly, regenerated cellulosic fibers such as viscose rayon and cuprammonium rayon, modified cellulosic fibers, such as cellulose acetate, or synthetic fibers such as those derived from polyesters, polyamides, polyacrylics, etc., alone or in combination with one another, may likewise be used. Blends of one or more of the above fibers may also be used if so desired.

In one embodiment of the present invention, the absorbent composites comprise a mixture of superabsorbent material and fibrous materials, wherein the WRV of the fibrous material is greater than about 0.2 g/g. Desirably, the WRV of the fibrous material is greater than about 0.35 g/g. More desirably, the WRV of the fibrous material is greater than about 0.5 g/g. Even more desirably, the WRV of the fibrous material is greater than about 0.7 g/g. Most desirably, the WRV of the fibrous material is greater than about 0.9 g/g. Table 6 contains WRV data for a variety of fibers.

TABLE 6
WRV of Various Fibers

| | Type of Fiber | WRV (g/g) |
|---|---|---|
| Regular Coosa Pulp | CR-54* | 1.12 |
| Coosa Pulp After Bivis Refiner | CR-54 Bivis# | 0.94 |
| Crosslinked CR-54 Bivis | CR-28# | 0.34 |
| Crosslinked CR-54 Bivis | CR-29# | 0.34 |
| Crosslinked, Debonder Treated | HBAFF** | 0.51 |
| Similar to HBAFF, No Debonder | NHB416** | 0.54 |
| Debonder Treated | HBAS** | 0.46 |
| Crosslinked | Curly Q*** | 0.42 |

*Alliance Forest Products (Coosa Pines, AL)
#KC Modified pulp
**Weyerhaeuser Company (Federal Way, Washington)
***Removed from Proctor & Gamble product

In one embodiment, the relative amount of superabsorbent material and fibrous material used to produce the absorbent composites of the present invention may vary depending on the desired properties of the resulting product, and the application of the resulting product. Desirably, the amount of Class I superabsorbent material in the absorbent composite is from about 20 wt % to about 95 wt % and the amount of fibrous material is from about 80 wt % to about 5 wt %, based on the total weight of the absorbent composite. More desirably, the amount of Class I superabsorbent material in the absorbent composite is from about 30 wt % to about 90 wt % and the amount of fibrous material is from about 70 wt % to about 10 wt %, based on the total weight of the absorbent composite. Most desirably, the amount of Class I superabsorbent material in the absorbent composite is from about 40 wt % to about 80 wt % and the amount of fibrous material is from about 60 wt % to about 20 wt %, based on the total weight of the absorbent composite.

In another embodiment, the basis weight of Class I superabsorbent material used to produce the absorbent composites of the present invention may vary depending on the desired properties, such as total composite thickness and basis weight, in the resulting product, and the application of the resulting product. For example, absorbent composites for use in infant diapers may have a lower basis weight and thickness compared to an absorbent composite for an incontinence device. Desirably, the basis weight of Class I superabsorbent material in the absorbent composite is greater than about 80 grams per square meter (gsm). More desirably, the basis weight of Class I superabsorbent material in the absorbent composite is from about 80 gsm to about 800 gsm. More desirably, the basis weight of Class I superabsorbent material in the absorbent composite is from about 120 gsm to about 700 gsm. Most desirably, the basis weight of Class I superabsorbent material in the absorbent composite is from about 150 gsm to about 600 gsm.

The absorbent composites of the present invention may be made by any process known to those of ordinary skill in the art. In one embodiment of the present invention, superabsorbent particles are incorporated into an existing fibrous substrate. Suitable fibrous substrates include, but are not limited to, nonwoven and woven fabrics. In many embodiments, particularly personal care products, preferred substrates are nonwoven fabrics. As used herein, the term “nonwoven fabric” refers to a fabric that has a structure of individual fibers or filaments randomly arranged in a mat-like fashion. Nonwoven fabrics may be made from a variety of processes including, but not limited to, air-laid processes, wet-laid processes, hydroentangling processes, staple fiber carding and bonding, and solution spinning. The superabsorbent material may be incorporated into the fibrous substrate as a solid particulate material or formed in situ from a solution applied to the substrate. The superabsorbent materials may be in any form suitable for use in absorbent composites including particles, fibers, flakes, spheres, and the like.

In a further embodiment of the present invention, the superabsorbent material and fibrous material are simultaneously mixed to form an absorbent composite. Desirably, the composite materials are mixed by an air-forming process known to those of ordinary skill in the art. Air-forming the mixture of fibers and superabsorbent material is intended to encompass both the situation wherein preformed fibers are air-laid with the superabsorbent material, as well as, the situation in which the superabsorbent material is mixed with the fibers as the fibers are being formed, such as through a meltblowing process.

In should be noted that the superabsorbent material may be distributed uniformly within the absorbent composite or may be non-uniformly distributed within the absorbent composite. The superabsorbent material may be distributed throughout the entire absorbent composite or may be distributed Within a small, localized area of the absorbent composite.

The absorbent composites of the present invention may be formed from a single layer of absorbent material or multiple layers of absorbent material. In the case of multiple layers, the layers may be positioned in a side-by-side or surface-to-surface relationship and all or a portion of the layers may be bound to adjacent layers. In those instances where the absorbent composite includes multiple layers, the entire thickness of the absorbent composite may contain one or more superabsorbent materials or each individual layer may separately contain some or no superabsorbent materials. Each individual layer may also contain different superabsorbent materials from an adjacent layer.

The absorbent composites according to the present invention are suited to absorb many fluids including body fluids such as urine, menses, and blood, and are suited for use in absorbent garments such as diapers, adult incontinence products, bed pads, and the like; in catamenial devices such as sanitary napkins, tampons, and the like; and in other absorbent products such as wipes, bibs, wound dressings, food packaging, and the like. Accordingly, in another aspect, the present invention relates to a disposable absorbent garment comprising an absorbent composite as described above. A wide variety of absorbent garments are known to those skilled in the art. The absorbent composites of the present invention can be incorporated into such known absorbent garments. Exemplary absorbent garments are generally described in U.S. Pat. Nos. 4,710,187 issued Dec. 1, 1987, to Boland et al.; 4,762,521 issued Aug. 9, 1988, to Roessler et al.; 4,770,656 issued Sep. 13, 1988, to Proxmire et al.; 4,798,603 issued Jan. 17, 1989; to Meyer et al.; which references are incorporated herein by reference.

As a general rule, the absorbent disposable garments according to the present invention comprise a body-side liner adapted to contact the skin of a wearer, an outer cover superposed in facing relation with the liner, and an absorbent composite, such as those described above, superposed on said outer cover and located between the body-side liner and the outer cover.

Those skilled in the art will readily understand that the superabsorbent materials and absorbent composites of the present invention may be advantageously employed in the preparation of a wide variety of products, including but not limited to, absorbent personal care products designed to be contacted with body fluids. Such products may only comprise a single layer of the absorbent composite or may comprise a combination of elements as described above. Although the superabsorbent materials and absorbent composites of the present invention are particularly suited for personal care products, the superabsorbent materials and absorbent composites may be advantageously employed in a wide variety of consumer products.

### Test Methods

For Testing Superabsorbent Materials

The methods for performing the Gel Bed Permeability (GBP) test and the Absorbency Under Load (AUL) test, used to distinguish Class I superabsorbent materials from Class II superabsorbent materials, are described below. Further, the pH test method is described below.

Gel Bed Permeability (GBP)

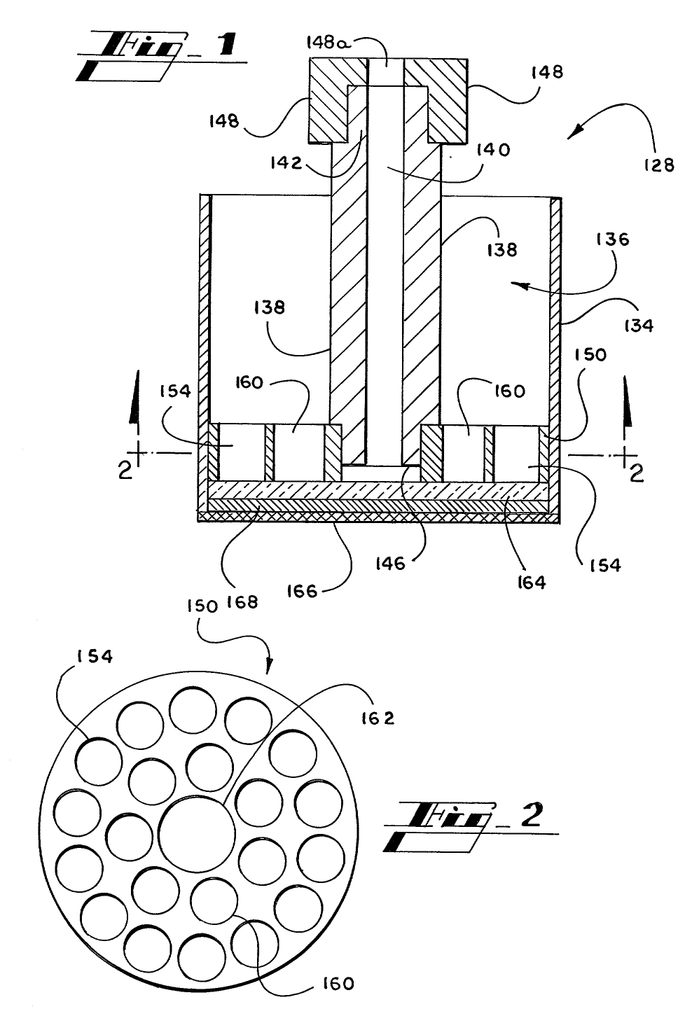

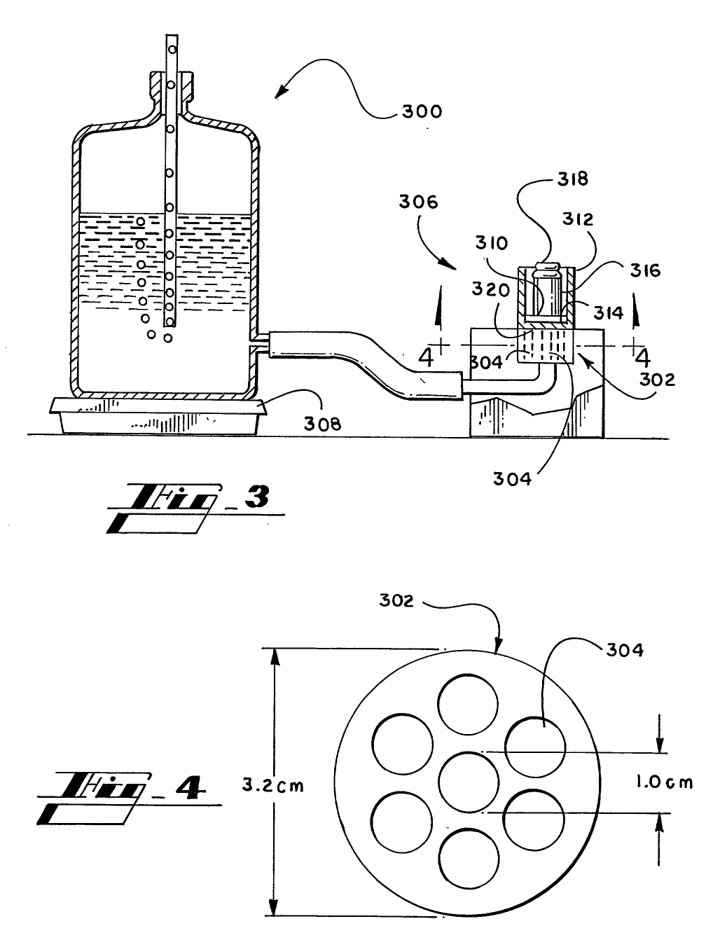

A suitable piston/cylinder apparatus for performing the GBP test is shown in FIGS. 1 and 2. Referring to FIG. 1, apparatus 128 consists of a cylinder 134 and a piston generally indicated as 136. As shown in FIG. 1, piston 136 consists of a cylindrical LEXAN® shaft 138 having a concentric cylindrical hole 140 bored down the longitudinal axis of the shaft. Both ends of shaft 138 are machined to provide ends 142 and 146. A weight, indicated as 148, rests on end 142 and has a cylindrical hole 148a bored through the center thereof. Inserted on the other end 146 is a circular piston head 150. Piston head 150 is sized so as to vertically move inside cylinder 134. As shown in FIG. 2, piston head 150 is provided with inner and outer concentric rings containing seven and fourteen approximately 0.375 inch (0.95 cm) cylindrical holes, respectively, indicated generally by arrows 160 and 154. The holes in each of these concentric rings are bored from the top to bottom of piston head 150. Piston head 150 also has cylindrical hole 162 bored in the center thereof to receive end 146 of shaft 138.

Attached to the bottom end of cylinder 134 is a No. 400 mesh stainless steel cloth screen 166 that is biaxially stretched to tautness prior to attachment. Attached to the bottom end of piston head 150 is a No. 400 mesh stainless steel cloth screen 164 that is biaxially stretched to tautness prior to attachment. A sample of superabsorbent material indicated as 168 is supported on screen 166.

Cylinder 134 is bored from a transparent LEXAN® rod or equivalent and has an inner diameter of 6.00 cm (area=28.27 cm²), a wall thickness of approximately 0.5 cm, and a height of approximately 5.0 cm. Piston head 150 is machined from a LEXAN® rod. It has a height of approximately 0.625 inches (1.59 cm) and a diameter sized such that it fits within cylinder 134 with minimum wall clearances, but still slides freely. Hole 162 in the center of the piston head 150 has a threaded 0.625 inch (1.59 cm) opening (18 threads/inch) for end 146 of shaft 138. Shaft 138 is machined from a LEXAN® rod and has an outer diameter of 0.875 inches (2.22 cm) and an inner diameter of 0.250 inches (0.64 cm). End 146 is approximately 0.5 inches (1.27 cm) long and is threaded to match hole 162 in piston head 150. End 142 is approximately 1 inch (2.54 cm) long and 0.623 inches (1.58 cm) in diameter, forming an annular shoulder to support the stainless steel weight 148. The annular stainless steel weight 148 has an inner diameter of 0.625 inches (1.59 cm), so that it slips onto end 142 of shaft 138 and rests on the annular shoulder formed therein. The combined weight of piston 136 and weight 148 equals approximately 596 g, which corresponds to a pressure of 0.30 psi (20,685 dynes/cm²) for an area of 28.27 cm².

When solutions flow through the piston/cylinder apparatus, the cylinder 134 generally rests on a 16 mesh rigid stainless steel support screen (not shown) or equivalent.

The piston and weight are placed in an empty cylinder to obtain a measurement from the bottom of the weight to the top of the cylinder. This measurement is taken using a caliper readable to 0.01 mm. This measurement will later be used to calculate the height of the gel bed. It is important to measure each cylinder empty and keep track of which piston and weight were used. The same piston and weight should be used for measurement when gel is swollen.

The superabsorbent layer used for GBP measurements is formed by swelling approximately 0.9 g of a superabsorbent material in the GBP cylinder apparatus (dry polymer should be spread evenly over the screen of the cylinder prior to swelling) with 0.9% (w/v) aqueous NaCl for a time period of about 60 minutes. The sample is taken from superabsorbent material which is prescreened through U.S. standard #30 mesh and retained on U.S. standard #50 mesh. The superabsorbent material, therefore, has a particle size of between 300 and 600 microns. The particles may be pre-screened by hand or automatically pre-screened with, for example, a Ro-Tap Mechanical Sieve Shaker Model B available from W. S. Tyler, Inc., Mentor, Ohio.

At the end of this period, the cylinder is removed from the fluid and the piston weight assembly is placed on the gel layer. The thickness of the swollen superabsorbent layer is determined by measuring from the bottom of the weight to the top of the cylinder with a micrometer. The value obtained when taking this measurement with the empty cylinder is subtracted from the value obtained after swelling the gel. The resulting value is the height of the gel bed H.

The GBP measurement is initiated by adding the NaCl solution to cylinder 134 until the solution attains a height of 4.0 cm above the bottom of superabsorbent layer 168. This solution height is maintained throughout the test. The quantity of fluid passing through superabsorbent layer 168 versus time is measured gravimetrically. Data points are collected every second for the first two minutes of the test and every two seconds for the remainder. When the data are plotted as quantity of fluid passing through the bed versus time, it becomes clear to one skilled in the art when a steady flow rate has been attained. Only data collected once the flow rate has become steady is used in the flow rate calculation. The flow rate, Q, through the superabsorbent layer 168, is determined in units of gm/sec by a linear least-square fit of fluid passing through the superabsorbent layer 168 (in grams) versus time (in seconds).

Permeability in cm² is obtained by the following equation:

K=[Q*H*Mu)]/[A*Rho*P]

where K=Gel Bed Permeability (cm²); Q=flow rate (g/sec); H=height of gel bed (cm); Mu=liquid viscosity (poise); A=cross-sectional area for liquid flow (cm²); Rho=liquid density (g/cm³); and P=hydrostatic pressure (dynes/cm²) [normally 3923 dynes/cm²].

Absorbency Under Load (A UL) test

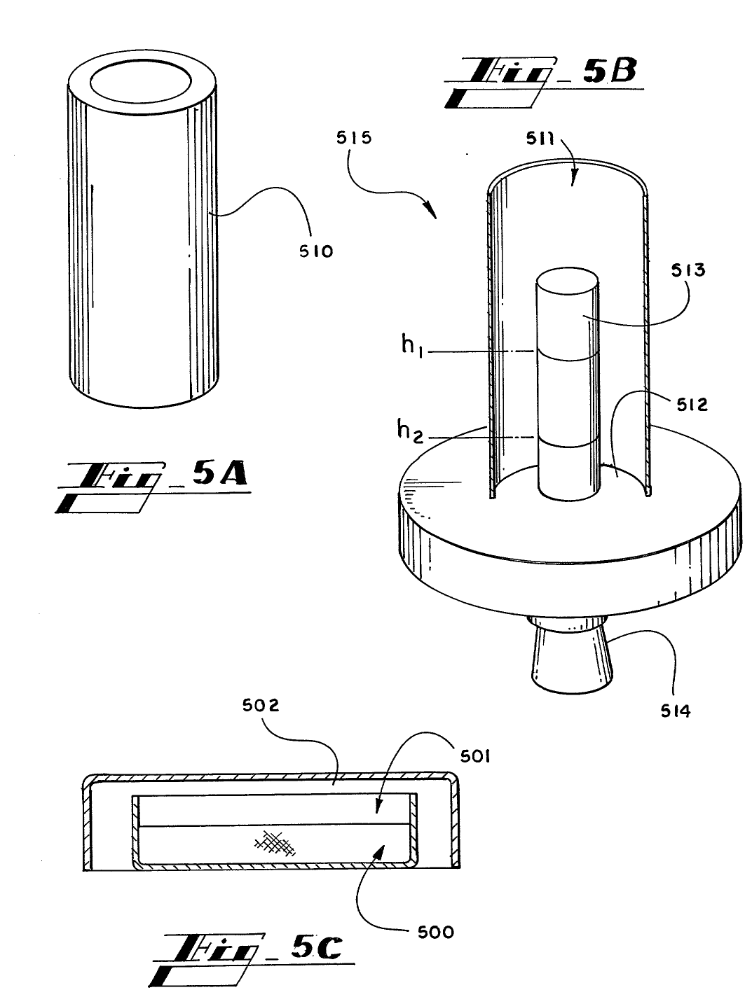

The Absorbency Under Load (AUL) test is a measure of the ability of a superabsorbent material to absorb a liquid while the superabsorbent material is under a restraining load. The test may best be understood by reference to FIGS. 3 and 4. Referring to FIG. 3, a demand absorbency tester (DAT) 300 is used, which is similar to a GATS (gravimetric absorbency test system), available from M/K Systems, Danners, Mass., as well as a system described by Lichstein in pages 129-142 of the INDA Technological Symposium Proceedings, March 1974.

A porous plate 302 is used having ports 304 confined within the 2.5 centimeter diameter covered, in use, by the Absorbency Under Load apparatus 306. FIG. 4 shows a cross-sectional view of porous plate 302. The porous plate 302 has a diameter of 3.2 centimeters with 7 ports (holes) 304 each with diameter of 0.30 centimeters. The porous plate 302 has one hole 304 in the center and the holes are spaced such that the distance from the center of one hole to another adjacent to it is 1.0 centimeter. An electrobalance 308 is used to measure the flow of the test fluid (an aqueous solution containing 0.9% w/v NaCl) into the superabsorbent material 310.

The AUL apparatus 306 used to contain the superabsorbent material may be made from 1 inch (2.54 centimeter), inside diameter, thermoplastic tubing 312 machined-out slightly to be sure of concentricity. One hundred mesh stainless steel wire cloth 314 is adhesively attached to the bottom of tubing 312. Alternatively, the steel wire cloth 314 may be heated in a flame until red hot, after which the tubing 312 is held onto the cloth until cooled. Care should be taken to maintain a flat, smooth bottom and not distort the inside of the tubing 312. A 4.4 gram piston 316 may be made from 1 inch (2.54 cm) solid material (e.g., Plexiglas) and machined to closely fit, without binding, in the tubing 312. A 200 gram weight 318 (outer diameter 0.98 inch (2.49 cm)) is used to provide 39,500 dynes per square centimeter (about 0.57 psi) restraining load on the superabsorbent material. For the purpose of the present invention, the pressure applied during the AUL test is referred to as 0.6 psi.

Desirably, about 0.160 grams of superabsorbent is used. The sample is taken from superabsorbent material, which is pre-screened through U.S. standard #30 mesh and retained on U.S. standard #50 mesh. The superabsorbent material, therefore, has a particle size of between 300 and 600 microns. The particles may be pre-screened by hand or automatically pre-screened with, for example, a Ro-Tap Mechanical Sieve Shaker Model B available from W. S. Tyler, Inc., Mentor, Ohio.

The desired amount of superabsorbent material 310 (0.160 grams) is weighed onto weigh paper and placed on the wire cloth 314 at the bottom of the tubing 312. The tubing 312 is shaken to level the superabsorbent material on the wire cloth 314. Care is taken to be sure no superabsorbent material is clinging to the wall of the tubing 312. The piston 316 and weight 318 are carefully placed on the superabsorbent material to be tested. The test is initiated by placing a 3 centimeter diameter glass filter paper 320 (Whatman filter paper Grade GF/A, available from Whatman International Ltd., Maidstone, England) onto the plate 302 (the paper is sized to be larger than the internal diameter and smaller than the outside diameter of the tubing 312) to ensure good contact, while eliminating evaporation over the ports 304 of the demand absorbency tester 300 and then allowing saturation to occur. The device is started by placing the apparatus 306 on the glass filter paper 320 and allowing saturation to occur. The amount of fluid picked up is monitored as a function of time either directly by hand, with a strip chart recorder, or directly into a data acquisition or personal computer system.

The amount of fluid pick-up measured after 60 minutes is the AUL value and is reported in grams of test liquid absorbed per gram of superabsorbent material as determined before starting the test procedure. A check can be made to ensure the accuracy of the test. The apparatus 306 can be weighed before and after the test with a difference in weight equaling the fluid pick-up.

pH Test Method

The pH test method used to determine the pH of superabsorbent materials of the present invention is performed as follows in a room having a room temperature of 23+/−1° C. (73.4 +/−1.8° F.) and a relative humidity of 50+/−2%. Into a 250 ml beaker with magnetic stirrer is added 150 g of a 0.9 wt % NaCl solution. The NaCl solution is stirred to create a vortex of about 2 inches. A 1.0 g sample of superabsorbent material, having a particle size of 300 to 600 microns, is weighed onto a weighing paper. The particles may be pre-screened by hand or automatically pre-screened with, for example, a Ro-Tap Mechanical Sieve Shaker Model B available from W. S. Tyler, Inc., Mentor, Ohio. The 300-600 micron particles are placed in a sealed container immediately to maintain its moisture content.

The superabsorbent material is slowly poured into the NaCl solution. The solution is allowed to stir for three minutes. After three minutes, the stirring is stopped and the magnetic stirrer is removed from the solution using clean tweezers. The beaker is then covered with a moisture barrier film (e.g., Parafilm®, available from Fischer-Scientific Company, Pittsburg, Pa.) and allowed to sit undisturbed for twenty minutes. As the superabsorbent material gels, it settles to the bottom of the beaker.

The electrodes of a pH meter (e.g., pH Meter Model 140, available from Corning, Corning, N.Y.) are rinsed with distilled water. The electrodes are inserted into the salt solution, making sure that the electrodes are not inserted into the sediment at the bottom of the beaker. The pH value is allowed to stabilize. The pH value is recorded, rounding the number to one decimal place.

For Testing Absorbent Composites

The test methods for the Composite Permeability Test, the Fluid Intake Flowback Evaluation test, and the Intake/Desorption test are described below:

Composite Permeability Test

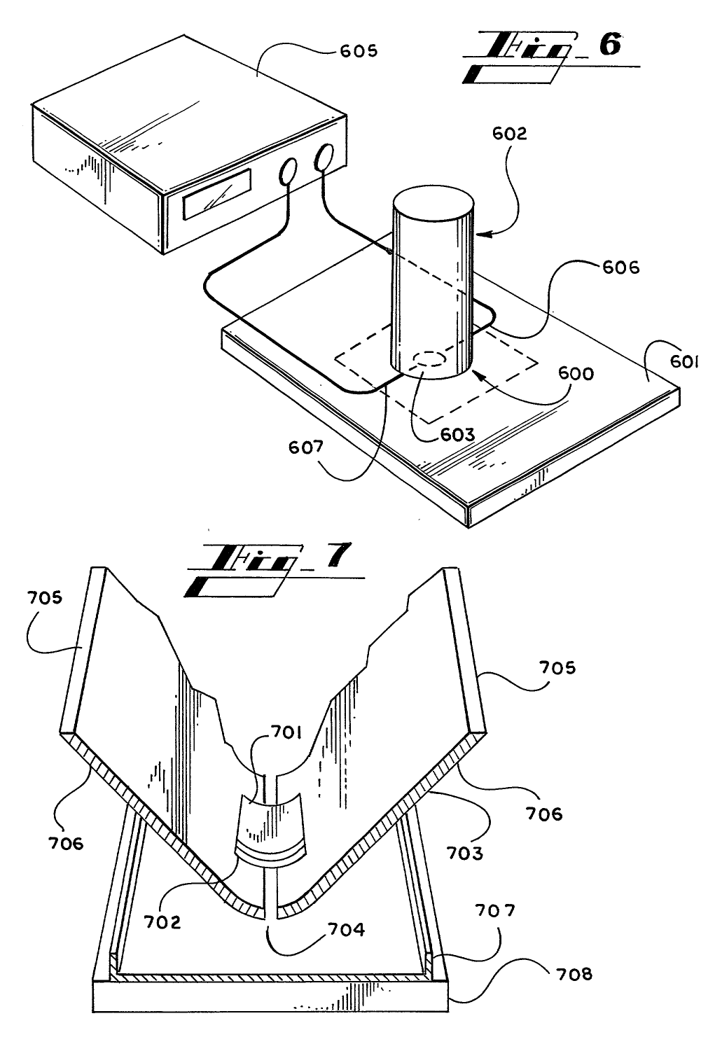

The Composite Permeability test determines the permeability of a composite in cm² by calculating the time for a fluid to flow through a composite. As shown in FIGS. 5a and 5b, the permeability tester consists of two Plexiglas or polycarbonate concentric cylinders, wherein one fits inside the other with very little clearance, but still slides freely. The inner cylinder 510 has an outer diameter of 6.9 cm and an inner diameter of 5.10 cm. The outer cylinder/base & stopper assembly 515 has a metal screen 512, on which the test material is placed for testing. This screen is desirably a type 504 stainless steel screen with a hole diameter of 0.156 inches (0.40 cm) and 63% open area, 20 gauge, and 3/16 inch (0.48 cm) center to center spacing. The outer cylinder 511 of the base and stopper assembly has an inner diameter of 7.0 cm and an outer diameter of 7.5 cm. A ruler 513 is on the outside of the outer cylinder 511 with height markings 3⅝ inch (9.21 cm) and 1⅛ inch (2.86 cm) from the bottom of the screen 512.

An absorbent composite of superabsorbent material and fluff, or fluff alone, is air-formed on tissue to a desired basis weight and density. This composite 500 is die cut to a desired size, desirably, a 6.83 cm (2.69 inch) diameter circle is used. As shown in FIG. 5c, the composite is placed in a dish 501 of approximately the same size (diameter) as the composite 500. This prevents swelling in the radial direction. The sample is saturated using a 0.9% (w/v) aqueous NaCl solution. A cover 502 is placed over the dish and allowed to sit 30 minutes to equilibrate. More solution may be added, if necessary, to fully saturate the sample. One will generally know when the composite is fully saturated when an excess of liquid exists within the dish 501. After a total of 30 minutes, the composite 500 and dish 501 are placed upside down on an absorbent medium such as paper toweling to remove the interstitial liquid. This is done by placing the paper toweling over the dish and composite, and while holding the dish and toweling, flipping it over. This puts the composite in direct contact with the toweling. No pressure is applied during this process.

After the blotting process, a wet bulk of the sample is taken by placing the sample under a thickness gauge with an acrylic platen or the like, which applies approximately 0.05 psi (3,448 dynes/cm²) pressure. The composite is then placed on the inner cylinder 510 and the outer cylinder (permeability tester) 515 is turned upside down over the inner cylinder with the composite.

The entire apparatus, which now contains the test composite and the inner cylinder, is flipped back over for the test. This ensures that the composite rests neatly (with least amount of handling) on o0 screen 512 at the bottom of test apparatus 515. The test fluid is poured in the inner cylinder on top of the composite. The fluid should be above the top mark on the ruler (at least 1 inch (2.54 cm)). before starting the test. To initiate the test, the stopper 514 is removed from the bottom of the permeability apparatus 515 and the timer is started when the fluid front reaches the top mark on the ruler (3⅝ inch (9.21 cm) above the screen) and the timer is stopped when the fluid front reaches the bottom mark on the ruler (1⅛ inch (2.86 cm) above the screen). Time in seconds is recorded.

Permeability (K) in cm² is calculated as follows:

K={[(ln (h₁/h₂)*Mu)/(g*Rho)]*WB/t}

where K=composite permeability (cm²); h₁=height of upper marker (cm) [normally 9.21 cm]; h₂=height of lower marker (cm) [normally 2.86 cm]; Mu=liquid viscosity (poise) [normally 0.01 poise]; g=acceleration due to gravity (cm/sec²) [normally 980 cm/sec²]; Rho=liquid density (gm/cm³) [normally 1.0 gm/cm³]; WB=wet bulk of composite (cm); t=time for liquid to move from h₁ to h₂ while flowing through composite (sec).

Fluid Intake Flowback Evaluation test

The Fluid Intake Flowback Evaluation (FIFE) test determines the amount of time required for an absorbent composite to intake a preset amount of fluid. A suitable apparatus for performing the FIFE test is shown in FIG. 6.

A composite of superabsorbent and fluff, or fluff only, is air-formed on tissue to a desired basis weight and density. The composite is cut to the desired size, in this case, the composite 600 is cut to a 5 inch (12.70 cm) square. The composite 600 is placed under the FIFE test pad 601. The test pad is a flexible conformable silicon bed that is 10 inches (25.4 cm) by 20 inches (50.8 cm). The silicon pad is constructed using Dow Corning 527 primeness silicon dielectric gel and wrapping it in shrinkable plastic wrapping. This pad is made with a sufficient thickness to produce a pressure of approximately 0.03 psi (2,069 dynes/cm²). The pad contains a Plexiglas cylinder 602 with an inner diameter of 5.1 cm and an outer diameter of 6.4 cm and the bottom of the cylinder has a cap 603 with a 1 inch (2.54 cm) circle bore in the center where the test fluid comes in direct contact with the composite 600. The center of the cylinder is located 6.75 inches (17.15 cm) down from the top edge of the silicon pad 601 and is centered from side to side (5 inches (12.70 cm) from the edge). An automated controller 605 can be connected to electrodes 606 and 607 that auto-initiate the test upon the entry of the test fluid. This can eliminate tester variability. The test fluid is desirably a 0.9% (w/v) NaCl solution.

The test is run by placing the composite 600 under the silicon test pad 601. The desired amount of fluid is dispensed from a positive displacement pump. The fluid amount in this case is calculated according to the composition of the composite. For example, the fluid amount for a 400 gsm composite of size 5 inch (12.70 cm) square consisting of 50% superabsorbent and 50% fluff is calculated by assuming the superabsorbent capacity is 30 g/g and the fluff capacity is 6 g/g. The total amount of capacity of the composite in grams is calculated and 25% of this amount is one insult. The fluid is dispensed at a rate of approximately 10 ml/sec. The time in seconds for fluid to drain from the cylinder 602 is recorded.

After a 15 minute wait, a second insult is done and after another 15 minute wait, the third and final insult is done. The FIFE Intake Rate for each insult is determined by dividing the insult fluid amount in milliliters by the time necessary for the fluid to drain from the cylinder 602 in seconds.

If during the test, leakage of fluid occurs from the top, bottom, or sides of the composite, the amount of leaked fluid should be measured. In this case, the FIFE Intake Rate for each insult is determined by subtracting the leaked fluid amount from the insult fluid amount and then dividing this quantity by the time for the fluid to drain from the cylinder 602 in seconds.

Intake/Desorption Test

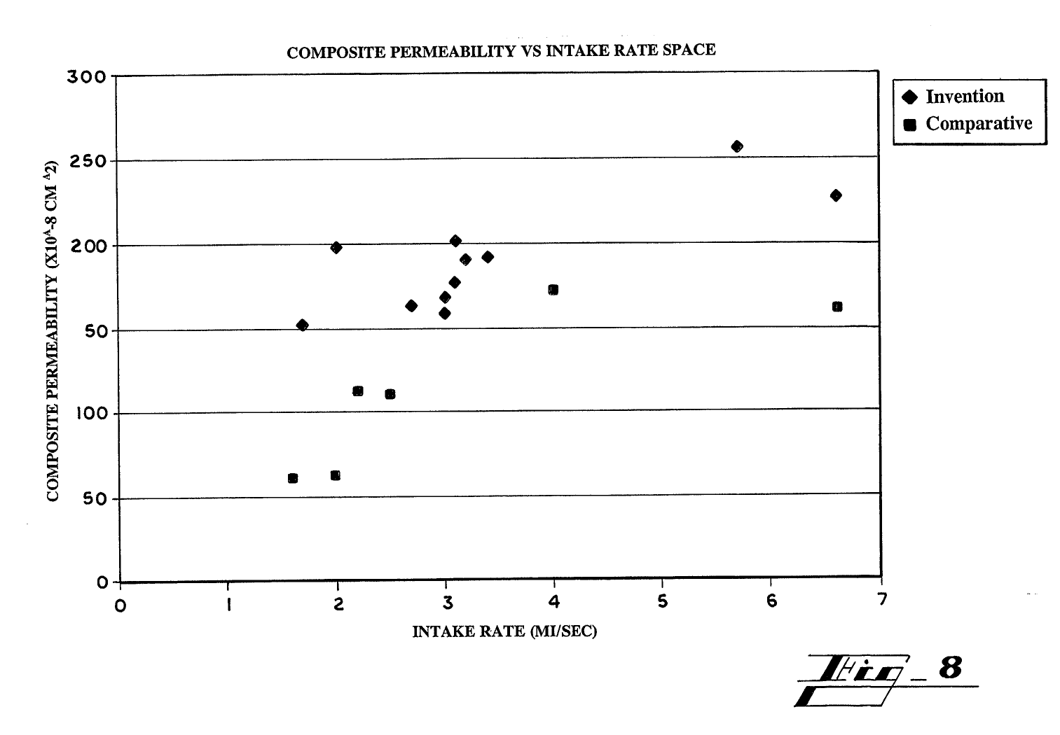

The Intake/Desorption test measures the intake and desorption capability of a material or composite. A suitable apparatus for performing the Intake/Desorption test is shown in FIG. 7.

A composite may consist of superabsorbent material and fluff, or fluff only. In this case, composites consisting of superabsorbent material and fluff were air-formed on tissue to a desired basis weight and density. The composite is then cut to the desired size, in this case, the composite is cut to 2.5 inches (6.35 cm) by 6 inches (15.24 cm). The dry weight of the composite 701 to be tested is recorded. The test composite 701 is placed on a piece of polyethylene film 702 that is the exact size of the test composite 701 and centered in a Plexiglas cradle 703 such that the length of the composite (15.24 cm) is perpendicular to the slot 704 in the bottom of the cradle 703. The cradle 703 has a width of 33 cm. The ends 705 of the cradle 703 are blocked off at a height of 19 cm to form an inner distance of 30.5 cm and an angle between the upper arms of 60 degrees between upper arms 706 of cradle 703. The cradle 703 has a 6.5 mm wide slot 704 at the lowest point running the length of the cradle 703. The slot 704 allows run-off from the test composite 701 to enter tray 707. The amount of run-off is recorded by a balance 708 readable to the nearest 0.01 g. A pre-set amount of liquid is delivered in the center of the test composite 701 at a desired rate. In this case the amount is 100 ml at a rate of 15 ml/sec and ½ inch (1.27 cm) above the sample. The amount of run-off is recorded.

The test composite 701 is immediately removed from the cradle 703 and placed on a 2.5 inches (6.35 cm) by 6 inches (15.24 cm) pre-weighed dry pulp/superabsorbent desorption pad having a total basis weight of 500 gsm and a density of about 0.20 g/cc and a superabsorbent material wt % of 60 in a horizontal position under 0.05 psi pressure for 15 minutes. The superabsorbent material is desirably Favor 880, available from Stockhausen, Inc. (Greensboro, N.C.). The pulp is desirably Coosa 1654, available from Alliance Forest Products (Coosa Pines, Ala.). This pressure is applied by using a Plexiglas plate. After the 15 minutes, the desorption pad weight is recorded and the test composite 701 is placed back in the cradle 703 and a second insult of 100 ml is done. After the amount of run-off is recorded, the test composite 701 is once again placed on a pre-weighed dry desorption pad under 0.05 psi (dynes/cm²) load for 15 minutes. After 15 minutes, a weight of the desorption pad is recorded. The composite 701 is placed back in the cradle 703 for a third insult. The amount of run-off is recorded and the test composite 701 is placed on a dry pre-weighed desorption pad under 0.05 psi pressure for 15 minutes. The amount of fluid picked up in g/g for each insult is calculated by subtracting the run-off from 100 ml and dividing by the dry weight of the test composite 701. A particularly useful measure of the ability of a composite to exhibit superior fluid intake of multiple insults over the life of the composite is to divide the 3^{rd} insult pickup value by the Is^{st} insult pickup value.

The present invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

### Testing of Superabsorbents for Gel Bed Permeability (GBP) and Absorbency Under Load (A UL)

Using the above-described procedure for measuring Gel Bed Permeability (GBP) and Absorbency Under Load (AUL), GBP and AUL values of various superabsorbent materials (SAM) were determined. The results of the testing are given below in Table 7.

TABLE 7
GBP and AUL Values for Superabsorbent Materials

| | | Gel Bed | AUL |
|---|---|---|---|
| | | Permeability | 0.6 psi |
| SAM | SAM Identification | (×10⁻⁹ cm²) | (g/g) |
| A | Stockhausen W-65431 | 302 | 23.4 |
| B | Dow AFA-173-60B | 194 | 21 |
| C | Dow XU 40671.00 | 1552 | 19.5 |
| D | Dow XUS 40665.07 | 925 | 19.6 |
| E | Dow XZ | 630 | 23.7 |
| F | Dow XUS 40667.01 | 967 | 22.2 |
| G | Dow XU40669 | 26 | 16 |
| H | Stockhausen 880 (600-850 | 110 | 27.9 |
| | micron) | | |
| I | Dow AFA-173-60A | 150 | 16.9 |
| J | Stockhausen W-65406 | 175 | 26.8 |
| K | Stockhausen W-77553 | 962 | 28 |
| L | Stockhausen Favor 880 | 79 | 28.4 |
| M | Dow DryTech 2035 | 58 | 23.9 |

As shown in Table 7, superabsorbents A through F and I exhibit a GBP value greater than 70×10⁻⁹ cm² and an AUL value of less than 25 g/g. Superabsorbents G and H and J through M exhibit a GBP value less than 70×10⁻⁹ cm² and/or an AUL value of greater than 25 g/g.

### EXAMPLE 2

### Testing of Superabsorbents for pH

Using the above-described procedure for measuring pH, pH values of various superabsorbent materials (SAM) were determined. The results of the testing are given below in Table 8.

TABLE 8
pH Values for Superabsorbent Materials

| Superabsorbent Material | pH |
|---|---|
| Stockhausen W-65431 | 5.8 |
| Dow AFA-173-60B | 5.6 |
| Dow XUS 40665.07 | 5.3 |
| Dow XU 40671.00 | 5.7 |
| Dow XZ | 6.0 |
| Dow XUS 40667.01 | 6.7 |
| Dow AFA-173-60A | 6.1 |
| Stockhausen Favor 880 | 6.8 |
| Dow DryTech 2035 | 6.3 |

### EXAMPLE 3

### Testing of Absorbent Composites for Composite Permeability, 3^{rd} FIFE Intake Rate, and Intake/Desorption 3^{rd}/1^{st} Pickup

The superabsorbents tested in Example 1 were combined with fluffed pulp fibers (Coosa River CR-1654; available from Alliance Forest Products (Coosa Pines, Ala.) and formed into webs using conventional air-forming equipment. The weight percent of superabsorbent material and the basis weight of superabsorbent material was varied as shown in Table 9.

TABLE 9
Nonwoven Webs of Superabsorbent Material and Pulp Fibers

| | SAM | SAM Concentration | SAM Basis Weight |
|---|---|---|---|
| Sample | Number | (mass %) | (gsm) |
| 1 | A | 50 | 200 |
| 2 | B | 50 | 200 |
| 3 | C | 50 | 200 |
| 4 | D | 50 | 200 |
| 5 | E | 50 | 200 |
| 6 | F | 50 | 200 |
| C-1 | G | 50 | 200 |
| C-2 | H | 50 | 200 |
| 11 | I | 50 | 200 |
| C-4 | J | 50 | 200 |
| C-5 | K | 50 | 200 |
| C-6 | L | 50 | 200 |
| C-7 | M | 50 | 200 |
| 7 | D | 50 | 250 |
| 8 | D | 50 | 150 |
| C-8 | D | 50 | 100 |
| 9 | D | 60 | 240 |
| 10 | D | 40 | 160 |
| C-9 | D | 30 | 120 |
| C-10 | L | 50 | 250 |
| C-11 | L | 50 | 150 |
| C-12 | L | 50 | 100 |
| C-13 | L | 60 | 240 |
| C-14 | L | 40 | 160 |
| C-15 | L | 30 | 120 |

The composites identified as samples 1 to 11 and comparitive examples C-1 to C-2 and C-4 to C-15 were evaluated or more of the following: composite permeability, 3^{rd} FIFE Intake Rate, and Intake/Desorption 3^{rd}/1^{st} Pickup as described The results of these tests are shown in Table 10.

TABLE 10
Testing for Composite Permeability, 3^{rd} FIFE Intake
Rate, and Intake/Desorption 3^{rd}/1^{st} Pickup

| | Composite | | |
|---|---|---|---|
| | Permeability | 3^{rd} FIFE Intake Rate | Intake/Desorption 3^{rd}/1^{st} |
| Sample | (×10⁻⁸ cm²) | (ml/sec) | Pickup |
| 1 | 191 | 3.2 | 1 |
| 2 | 177 | 3.1 | 1.18 |
| 3 | 192 | 3.4 | 1.35 |
| 4 | 202 | 3.1 | 1.52 |
| 5 | 115 | 2.1 | 1.18 |
| 6 | 168 | 3.0 | 1.22 |
| 7 | — | 3.5 | — |
| 8 | — | 5.5 | — |
| 9 | 159 | 3.0 | — |
| 10 | 255 | 5.7 | — |
| C-1 | 163 | 2.7 | 0.92 |
| C-2 | 110 | 2.5 | 0.99 |
| 11 | 100 | 2.5 | 1.30 |
| C-4 | 198 | 2.0 | 0.86 |
| C-5 | 152 | 1.7 | 0.98 |
| C-6 | 112 | 2.2 | 0.90 |
| C-7 | 61 | 1.6 | 0.92 |
| C-8 | — | 4.4 | — |
| C-9 | 226 | 6.6 | — |
| C-10 | — | 2.5 | — |
| C-11 | — | 3.3 | — |
| C-12 | — | 4.4 | — |
| C-13 | 63 | 2.0 | — |
| C-14 | 172 | 4.0 | — |
| C-15 | 161 | 6.6 | — |

As can be seen when examining the above data, the Class I superabsorbent materials provided improved intake performance.

The above disclosed examples are preferred embodiments and are not intended to limit the scope of the present invention in any way. Various modifications and other embodiments and uses of the disclosed superabsorbent polymers, apparent to those of ordinary skill in the art, are also considered to be within the scope of the present invention.

What is claimed is:

1. An absorbent composite comprising from about 20 to about 95 weight percent superabsorbent material, based on the total weight of the composite, and from about 80 to about 5 weight percent fibers, based on the total weight of the composite; wherein the absorbent composite has a Composite Permeability (CP) value at full swelling and a 3^{rd} Insult Fluid Intake Flowback Evaluation (FIFE) intake rate (IR); and wherein the CP value and the IR value satisfy the following conditions:
when the IR value of the absorbent composite is greater than 0 ml/sec and less than about 3.00 ml/sec, the CP value is given by the following equation:
CP≧{135−[(35/3)×(3.00−IR)]}×10⁻⁸;
when the IR value of the absorbent composite is greater than about 3.00 ml/sec and less than about 3.70 ml/sec, the CP value is given by the following equation:
CP≧{175−[(400/7)×(3.70−IR)]}×10⁻⁸; and
when the IR value of the absorbent composite is greater than about 3.70 ml/sec, the CP value is greater than or equal to 175×10⁻⁸, wherein CP has units of cm².

2. The absorbent composite of claim 1, wherein the CP value and the IR value satisfy the following conditions:
when the IR value of the absorbent composite is greater than 0 ml/sec and less than about 3.00 ml/sec, the CP value is given by the following equation:
CP≧{150−[(35/3)×(3.00−IR)]}×10⁻⁸;
when the IR value of the absorbent composite is greater than about 3.00 ml/sec and less than about 3.70 ml/sec, the CP value is given by the following equation:
CP≧{190−[(400/7)×(3.70−IR)]}×10⁻⁸; and
when the IR value of the absorbent composite is greater than about 3.70 ml/sec, the CP value is greater than or equal to 190×10⁻⁸, wherein CP has units of cm².

3. The absorbent composite of claim 2, wherein the CP value and the IR value satisfy the following conditions:
when the IR value of the absorbent composite is greater than 0 ml/sec and less than about 3.00 ml/sec, the CP value is given by the following equation:
CP≧{165−[(35/3)×(3.00−IR)]}×10⁻⁸;
when the IR value of the absorbent composite is greater than about 3.00 ml/sec and less than about 3.70 ml/sec, the CP value is given by the following equation:
CP≧{205−[(400/7)×(3.70−IR)]}×10⁻⁸; and
when the IR value of the absorbent composite is greater than about 3.70 ml/sec, the CP value is greater than or equal to 205×10⁻⁸, wherein CP has units of cm².

4. The absorbent composite of claim 1, wherein the absorbent composite has a CP value at full swelling of greater than about 175×10⁻⁸ cm².

5. The absorbent composite of claim 1, wherein the absorbent composite has a CP value at full swelling of greater than about 190×10⁻⁸ cm².

6. The absorbent composite of claim 1, wherein the absorbent composite has a CP value at full swelling of greater than about 205×10⁻⁸ cm².

7. The absorbent composite of claim 1, wherein the absorbent composite has a CP value at full swelling of greater than about 225×10⁻⁸ cm².

8. The absorbent composite of claim 1, wherein the absorbent composite has a 3^{rd} Insult FIFE intake rate of about 2.00 ml/sec and a CP value at full swelling of greater than about 125×10⁻⁸ cm².

9. The absorbent composite of claim 1, wherein the absorbent composite has a 3^{rd} Insult FIFE intake rate of greater than about 0.1 ml/sec and a CP value at full swelling of greater than about 175×10⁻⁸ cm².

10. The absorbent composite of claim 1, wherein the absorbent composite has a 3^{rd} Insult FIFE intake rate of greater than about 2.50 ml/sec and a CP value at full swelling of greater than about 175×10⁻⁸ cm².

11. The absorbent composite of claim 1, wherein the absorbent composite has a 3^{rd} Insult FIFE intake rate of greater than about 3.00 ml/sec and a CP value at full swelling of greater than about 175×10⁻⁸ cm².

12. The absorbent composite of claim 1, wherein the absorbent composite comprises from about 30 to about 90 weight percent superabsorbent material and from about 70 to about 10 weight percent fibers.

13. The absorbent composite of claim 12, wherein the absorbent composite comprises from about 40 to about 80 weight percent superabsorbent material and from about 60 to about 20 weight percent fibers.

14. The absorbent composite of claim 1, wherein the fibers have a water retention value (WRV) greater than about 0.2 gig.

15. The absorbent composite of claim 14, wherein the fibers have a water retention value (WRV) greater than about 0.5 gig.

16. The absorbent composite of claim 15, wherein the fibers have a water retention value (WRV) greater than about 0.7 g/g.

17. The absorbent composite of claim 16, wherein the fibers have a water retention value (WRV) greater than about 0.9 gig.

18. The absorbent composite of claim 1, wherein the absorbent composite has a basis weight of sup erabsorbent material of greater than about 80 grams per square meter.

19. The absorbent composite of claim 18, wherein the absorbent composite has a basis weight of superabsorbent material of from about 80 grams per square meter to about 800 grams per square meter.

20. The absorbent composite of claim 19, wherein the absorbent composite has a basis weight of superabsorbent material of from about 120 grams per square meter to about 700 grams per square meter.

21. The absorbent composite of claim 20, wherein the absorbent composite has a basis weight of superabsorbent material of from about 150 grams per square meter to about 600 grams per square meter.

22. The absorbent composite of claim 1, wherein the superabsorbent material comprises a sodium polyacrylate.

23. The absorbent composite of claim 1, wherein the superabsorbent material has a Gel Bed Permeability (GBP) value of greater than about 70×10⁻⁹ cm² and an Absorbency Under Load (AUL) value at 0.6 psi of less than about 25 g/g.

24. The absorbent composite of claim 1, wherein the superabsorbent material has a GBP value of greater than about 70 ×10⁻⁹cm² and an AUL value at 0.6 psi of less than about 24 g/g.

25. The absorbent composite of claim 1, wherein the superabsorbent material has a GBP value of greater than about 70 ×10⁻⁹ cm² and an AUL value at 0.6 psi of less than about 23 g/g.

26. The absorbent composite of claim 1, wherein the superabsorbent material has a GBP value of greater than about 70 ×10⁻⁹ cm² and an AUL value at 0.6 psi of less than about 21 g/g.

27. The absorbent composite of claim 1, wherein the superabsorbent material has a GBP value of greater than about 150 ×10⁻⁹ cm² and an AUL value at 0.6 psi of less than about 25 g/g.

28. The absorbent composite of claim 1, wherein the superabsorbent material has a GBP value of greater than about 150 ×10⁻⁹ cm² and an AUL value at 0.6 psi of less than about 24 g/g.

29. The absorbent composite of claim 1, wherein the superabsorbent material has a GBP value of greater than about 150 ×10⁻⁹ cm² and an AUL value at 0.6 psi of less than about 23 g/g.

30. The absorbent composite of claim 1, wherein the superabsorbent material has a GBP value of greater than about 150 ×10⁻⁹ cm² and an AUL value at 0.6 psi of less than about 21 g/g.

31. The absorbent composite of claim 1, wherein the superabsorbent material has a GBP value of greater than about 250 ×10⁻⁹ cm² and an AUL value at 0.6 psi of less than about 25 g/g.

32. The absorbent composite of claim 1, wherein the superabsorbent material has a GBP value of greater than about 250 ×10⁻⁹ cm² and an AUL value at 0.6 psi of less than about 24 g/g.

33. The absorbent composite of claim 1, wherein the superabsorbent material has a GBP value of greater than about 250 ×10⁻⁹ cm² and an AUL value at 0.6 psi of less than about 23 g/g.

34. The absorbent composite of claim 1, wherein the superabsorbent material has a GBP value of greater than about 250 ×10⁻⁹ cm² and an AUL value at 0.6 psi of less than about 21 g/g.

35. The absorbent composite of claim 1, wherein the superabsorbent material has a pH value of from about 3 to about 8.

36. The absorbent composite of claim 1, wherein the superabsorbent material has a pH value of from about 4 to about 8.

37. The absorbent composite of claim 1, wherein the superabsorbent material has a pH value of from about 5.2 to about 8.

38. A method of making an absorbent composite, said method comprising:
forming an absorbent composite comprising from about 20 to about 95 weight percent superabsorbent material, based on the total weight of the composite, and from about 80 to about 5 weight percent fibers, based on the total weight of the composite; wherein the absorbent composite has a Composite Permeability (CP) value at full swelling and a 3^{rd} Insult Fluid Intake Flowback Evaluation (FIFE) intake rate (IR); and wherein the CP value and the IR value satisfy the following conditions:
when the IR value of the absorbent composite is greater than 0 ml/sec and less than about 3.00 ml/sec, the CP value is given by the following equation:
CP≧{135−[(35/3)×(3.00−IR)]}×10⁻⁸;
when the IR value of the absorbent composite is greater than about 3.00 ml/sec and less than about 3.70 ml/sec, the CP value is given by the following equation:
CP≧{175−[(400/7)×(3.70−IR)]}×10⁻⁸; and
when the IR value of the absorbent composite is greater than about 3.70 ml/sec, the CP value is greater than or equal to 175×10⁻⁸, wherein CP has units of cm².

39. The method of claim 38, wherein the CP value and the IR value satisfy the following conditions:
when the IR value of the absorbent composite is greater than 0 ml/sec and less than about 3.00 ml/sec, the CP value is given by the following equation:
CP≧{150−[(35/3)×(3.00−IR)]}×10⁻⁸;
when the IR value of the absorbent composite is greater than about 3.00 ml/sec and less than about 3.70 ml/sec, the CP value is given by the following equation:
CP≧{190−[(400/7)×(3.70−IR)]}×10⁻⁸; and
when the IR value of the absorbent composite is greater than about 3.70 ml/sec, the CP value is greater than or equal to 190×10⁻⁸, wherein CP has units of cm².

40. The method of claim 39, wherein the CP value and the IR value satisfy the following conditions:
when the IR value of the absorbent composite is greater than 0 ml/sec and less than about 3.00 ml/sec, the CP value is given by the following equation:
CP≧{165−[(35/3)×(3.00−IR)]}×10⁻⁸;
when the IR value of the absorbent composite is greater than about 3.00 ml/sec and less than about 3.70 ml/sec, the CP value is given by the following equation:
CP≧{205−[(400/7)×(3.70−IR)]}×10⁻⁸; and
when the IR value of the absorbent composite is greater than about 3.70 ml/sec, the CP value is greater than or equal to 205×10⁻⁸, wherein CP has units of cm².

41. The method of claim 38, wherein the absorbent composite is formed by an air-forming step.

42. The method of claim 38, wherein the absorbent composite has a basis weight of superabsorbent material of greater than about 80 grams per square meter.

43. The method of claim 38, wherein the superabsorbent material has a Gel Bed Permeability (GBP) value of greater than about 70×10⁻⁹cm² and an Absorbency Under Load (AUL) value at 0.6 psi of less than about 25 g/g.

44. A disposable garment comprising at least one absorbent composite, wherein the at least one absorbent composite comprises from about 20 to about 95 weight percent superabsorbent material, based on the total weight of the composite, and from about 80 to about 5 weight percent fibers, based on the total weight of the composite; wherein the absorbent composite has a Composite Permeability (CP) value at full swelling and a 3^{rd} Insult Fluid Intake Flowback Evaluation (FIFE) intake rate (IR); and wherein the CP value and the IR value satisfy the following conditions:
when the IR value of the absorbent composite is greater than 0 ml/sec and less than about 3.00 ml/sec, the CP value is given by the following equation:
CP≧{135−[(35/3)×(3.00−IR)]}×10⁻⁸;
when the IR value of the absorbent composite is greater than about 3.00 ml/sec and less than about 3.70 ml/sec, the CP value is given by the following equation:
CP≧{175−[(400/7)×(3.70−IR)]}10⁻⁸; and
when the IR value of the absorbent composite is greater than about 3.70 ml/sec, the CP value is greater than or equal to 175×10⁻⁸, wherein CP has units of cm².

45. The disposible garment of claim 44, wherein the CP value and the IR value satisfy the following conditions:
when the IR value of the absorbent composite is greater than 0 ml/sec and less than about 3.00 ml/sec, the CP value is given by the following equation:
CP≧{150−[(35/3)×(3.00−IR)]}×10⁻⁸;
when the IR value of the absorbent composite is greater than about 3.00 ml/sec and less than about 3.70 ml/sec, the CP value is given by the following equation:
CP≧{190−[(400/7)×(3.70−IR)]}×10⁻⁸; and
when the IR value of the absorbent composite is greater than about 3.70 ml/sec, the CP value is greater than or equal to 190×10⁻⁸, wherein CP has units of cm².

46. The disposible garment of claim 45, wherein the CP value and the IR value satisfy the following conditions:
when the IR value of the absorbent composite is greater than 0 ml/sec and less than about 3.00 ml/sec, the CP value is given by the following equation:
CP≧{165−[(35/3)×(3.00−IR)]}×10⁻⁸;
when the IR value of the absorbent composite is greater than about 3.00 ml/sec and less than about 3.70 ml/sec, the CP value is given by the following equation:
CP≧{205−[(400/7)×(3.70−IR)]}×10⁻⁸; and
when the IR value of the absorbent composite is greater than about 3.70 ml/sec, the CP value is greater than or equal to 205×10⁻⁸, wherein CP has units of cm².

47. The disposable garment of claim 44, wherein the superabsorbent material has a Gel Bed Permeability (GBP) value of greater than about 70×10⁻⁹ cm² and an Absorbency Under Load (AUL) value at 0.6 psi of less than about 25 g/g.
